(19) United States
(12) Patent Application Publication
(10) Pub. No.: US 2021/0322246 A1
(43) Pub. Date: Oct. 21, 2021

(54) MOTION ASSISTING DEVICE

(71) Applicant: TOYOFLEX CORPORATION, Tokyo (JP)

(72) Inventors: Yusaku FUJITA, Tokyo (JP); Daiki OGATA, Tokyo (JP)

(73) Assignee: TOYOFLEX CORPORATION, Tokyo (JP)

(21) Appl. No.: 17/362,153

(22) Filed: Jun. 29, 2021

Related U.S. Application Data
(63) Continuation of application No. PCT/JP2019/051578, filed on Dec. 27, 2019.

(30) Foreign Application Priority Data
Jan. 11, 2019 (JP) 2019-003405

(51) Int. Cl. A61H 1/02; B25J 9/00

(52) U.S. Cl. CPC A61H 1/024; B25J 9/0006; A61H 2201/1261; A61H 2201/1671; A61H 2201/1463; A61H 2201/165

(57) ABSTRACT

A motion assisting device includes a cord-like body that is wound around the outer peripheral surfaces of a first pulley and a second pulley. An elastic body energizing force in accordance with a rotation angle of the first pulley is provided to the first pulley at a first rotation axis, which is a rotation axis of the first pulley. Along with the rotation of the first pulley by energizing force of the elastic body, the cord-like body is wound around the first pulley and unwound from the second pulley, whereby the second pulley is rotated. Along with the rotation of the second pulley, a small gear rotating together with the second pulley with a second rotation axis as a center and a large gear fittable to the small gear rotate, and the large gear rotates a lower limb rod through a transmission mechanism.

### CROSS REFERENCE TO RELATED APPLICATIONS

This is a Continuation of Application No. PCT/JP2019/051578 filed Dec. 27, 2019, which claims priority to JP 2019-003405 filed Jan. 11, 2019. The disclosure of the prior applications is hereby incorporated by reference herein in its entirety.

### FIELD

The disclosed embodiments relate to a motion assisting device.

### BACKGROUND

Patent Literature 1 discloses a forward leaning posture support device including a compression coil spring arranged on the side of a femoral region of a human body, an upper body support frame supporting the upper body of the human body, and a wire arranged therebetween. The upper support frame is tilted to a base part so that the support device compresses the compression coil spring and then transmits energizing force in a direction lifting the human body to the upper body support frame.

However, in the support device described in Patent Literature 1, the cord-like body is connected directly to an elastic body of the device, and thus the assisting force to be exerted depends on the elastic force. This causes a problem of limiting the strength of assisting force.

### CITATION LIST

### Patent Literature

[Patent Literature 1] Japanese Patent Application Publication No. 2016-209443

### SUMMARY

The disclosed embodiments have been devised in consideration of such circumstances, and aim to provide a motion assisting device capable of generating a large assisting force and assisting a user in returning to the upright state.

To address these considerations, a motion assisting device according to the disclosed embodiments is, for example, a motion assisting device assisting a motion of lifting an upper body of a user, the motion assisting device including: an assisting unit which is mounted to a lateral side of the user, the assisting unit including: a first pulley having a first rotation axis and being rotatable with the first rotation axis as a center; an elastic body provided at the first rotation axis and energizing force in accordance with a rotation angle of the first pulley to the first pulley; a cord-like body including a first end connected to the first pulley; a second pulley to which a second end of the cord-like body is connected and that is rotatable with a second rotation axis as a center, the second pulley having an outer diameter larger than an outer diameter of the first pulley; a small gear provided at the second pulley and rotating together with the second pulley with the second rotation axis as a center; and a large gear having a larger number of teeth than the small gear, being fittable to the small gear, and being rotatable with a third rotation axis as a center, a lower limb rod that is connected to the third rotation axis and is attachable to a femoral region of the user; and a transmission mechanism that transmits rotation of the large gear to the lower limb rod or transmits rotation of the lower limb rod to the large gear, wherein, the cord-like body is wound around outer peripheral surfaces of the first pulley and the second pulley; and energizing force of the elastic body rotates the first pulley, so that the cord-like body is wound around the first pulley and the cord-like body is unwound from the second pulley, whereby the second pulley is rotated, the rotation of the second pulley rotates the small gear and the large gear, and the rotation of the large gear rotates the lower limb rod through the transmission mechanism.

In the motion assisting device, the cord-like body having the first end connected to the first pulley and the second end connected to the second pulley is wound around the outer peripheral surfaces of the first pulley and the second pulley, and an elastic body energizing force in accordance with a rotation angle of the first pulley to the first pulley is provided at the first rotation axis that is a rotation axis of the first pulley. Along with the rotation of the first pulley by energizing force of the elastic body, the cord-like body is wound around the first pulley and unwound from the second pulley, whereby the second pulley is rotated. Along with the rotation of the second pulley, the small gear rotating together with the second pulley with the second rotation axis as a center and the large gear fittable to the small gear rotate, and the large gear rotates the lower limb rod through the transmission mechanism. In this manner, it is possible to assist the user in returning to the upright state with a simple configuration without using a power supply. Moreover, the outer diameter of the second pulley is larger than the outer diameter of the first pulley, and the number of teeth of the large gear is larger than the number of teeth of the small gear. In this manner, it is possible to amplify the assisting force of the elastic body in two stages and generate large assisting force. That is, as compared with the configuration where the elastic body and the lower limb rod are connected directly by the cord-like body, the influence by the elastic force of the elastic body is smaller, allowing a torque to be larger or substantially constant.

Here, the transmission mechanism may perform switching between a first form and a second form in accordance with a relative angle of the lower limb rod and the large gear, the first form means that the lower limb rod does not rotate the large gear and the second form means that the lower limb rod applies force in a rotation direction to the large gear so as to rotate the large gear. In this manner, it is not necessary to switch whether or not a motion assistance is needed, allowing the user to perform his/her own action continuously. Moreover, when the user stands upright or in the state within a given angular range from the upright state (during walking), the lower limb rod idles in space. Thus, the user can walk smoothly.

Here, an angle for switching between the first form and the second form on the front side of the user may be equal to or larger than an angle for switching between the first form and the second form on the back side of the user. In this manner, the angle for switching between the first form and the second form can be an angle following the walking motion.

Here, the large gear includes a fan-shaped recess with the third rotation axis as a center on a surface to which the lower limb rod is connected; the recess is formed larger than a connection part with the large gear of the lower limb rod in a rotation direction of the lower limb rod; the lower limb rod is rotatable in the recess along a plane surface of a bottom part of the recess; and rotation of the large gear is transmittable to the lower limb rod, and rotation of the lower limb rod is transmittable to the large gear when the lower limb rod comes into contact with a side wall of the recess.

Here, the lower limb rod includes a pin on a surface facing the large gear; the large gear includes a long hole into which the pin is inserted; the pin is movable in the long hole in accordance with rotation of the lower limb rod; and rotation of the large gear is transmittable to the lower limb rod, and rotation of the lower limb rod is transmittable to the large gear, when the pin comes into contact with an end of the long hole.

Here, the motion assisting device further includes a second cord-like body connecting the lower limb rod and the large gear; wherein rotation of the large gear is transmittable to the lower limb rod, and rotation of the lower limb rod is transmittable to the large gear, when the lower limb rod rotates in a direction separating a fixing portion between the second cord-like body and the large gear from a fixing portion between the second cord-like body and the lower limb rod by a given distance or larger.

Here, the assisting unit includes: a first assisting unit that is attached to a left side of the user and a second assisting unit that is attached to a right side of the user, the motion assisting device may further include a chest fixing member that is fixed to a chest of the user, a waist fixing member that is fixed to a waist of the user, and a connection part that connects the first assisting unit, the second assisting unit, the chest fixing member, and the waist fixing member. In this manner, it is possible to suppress torsion of the assisting unit during assisting operation and allow the lower limb rod to rotate smoothly.

Here, the motion assisting device may further include an attaching member that is provided to be rotatable with the third rotation axis as a center, in which the waist fixing member is provided at the attaching member to be rotatable with a fourth rotation axis as a center, and the third rotation axis and the fourth rotation axis may be provided at mutually different positions. Thus, the rotary motion of one of the assisting unit and the waist fixing member is not transmitted directly to the other, thereby enabling the assisting unit and the waist fixing member to rotate independently. The assisting unit rotates in accordance with an angle of a femoral region, while the waist fixing member rotates in accordance with an angle of a waist part. In a human body, the rotation axis of the femoral region and the rotation axis of the waist are different in height. Therefore, if the height of the rotation axis of the assisting unit is arranged to be substantially the same as that of the rotation axis of the waist fixing member, it is not possible to follow the forward bending motion of a human body, causing torsion of the waist fixing member. On the other hand, if the height of the rotation axis of the chest fixing member is different from that of the rotation axis of the waist fixing member, it is possible to follow the forward bending motion of a human body.

Here, the elastic body is a spiral spring in which a plate-shaped member is wound in a surface direction into a spiral shape, the elastic body is provided at the first rotation axis, an outer periphery of the first pulley is gradually tapered toward a distal end, and the cord-like body is wound around an outer peripheral surface of the first pulley from a base of the first pulley toward a distal end of the first pulley. As the cord-like body wound around the outer periphery of the first pulley is drawn out, the elastic force of the elastic body is increased, and the outer diameter of the first pulley wound by the cord-like body is larger. Therefore, it is possible to keep force required to release the cord-like body from the first pulley (an apparent spring constant) constant.

Here, a helical first groove is provided on the outer periphery of the first pulley, a helical second groove is provided on the outer periphery of the second pulley, the cord-like body is stored in the first groove and the second groove, and the cord-like body may be substantially orthogonal to the first rotation axis and the second rotation axis when the first pulley and the second pulley are viewed from a side surface. In this manner, it is possible to prevent the cord-like body from being drawn out diagonally, smoothen sliding of the cord-like body and rotation of the first pulley and the second pulley, and suppress a loss in energizing force.

With the disclosed embodiments, it is possible to generate a large assisting force and assist a user in returning to the upright state.

### BRIEF DESCRIPTION OF THE DRAWINGS

FIG. 1 is a perspective view of a motion assisting device 1.

FIG. 2 is a front view of the motion assisting device 1.

FIG. 3 is a right side view of the motion assisting device 1.

FIG. 4 is a back view of the motion assisting device 1.

FIG. 5 is a left side view schematically illustrating an assisting unit 20.

FIG. 6 is a perspective view schematically illustrating a power unit 40.

FIG. 7 is a perspective view schematically illustrating a power unit 40.

FIGS. 8A-8D are diagrams schematically illustrating a large gear 46.

FIG. 9 is a diagram illustrating a state of the motion assisting device 1 (assisting unit 20) when a leg of the user is positioned rearward during walking.

FIG. 10 is a diagram illustrating a state of the motion assisting device 1 (assisting unit 20) when a waist of the user is slightly bent.

FIG. 11 is a diagram illustrating a state of the motion assisting device 1 (assisting unit 20) when the waist of the user is further bent.

FIGS. 12A-12B are diagrams illustrating states of a first pulley 42, a second pulley 44, and a cord-like body 43.

FIGS. 13A-13B are diagrams illustrating states of a first pulley 42, a second pulley 44, and a cord-like body 43.

FIGS. 14A-14D are diagrams schematically illustrating the first pulley 42.

FIGS. 15A-15D are diagrams schematically illustrating the second pulley 44.

FIGS. 16A-16B are longitudinal sectional views schematically illustrating a power unit 40A of a motion assisting device 2 according to a second embodiment.

FIG. 17 is a longitudinal sectional view schematically illustrating a power unit 40B of a motion assisting device 3 according to a third embodiment.

FIGS. 18A-18B are longitudinal sectional views schematically illustrating the power unit 40B of the motion assisting device 3 according to the third embodiment.

### DESCRIPTION OF EMBODIMENTS

Hereinafter, various disclosed embodiments will be described with reference to the drawings. The motion assisting device according to the disclosed embodiments assists a motion of lifting the upper body (stretching the waist) of a user with the use of a cord-like body.

### First Embodiment

The following will describe the motion assisting device 1 according to the first of the disclosed embodiments with reference to the drawings.

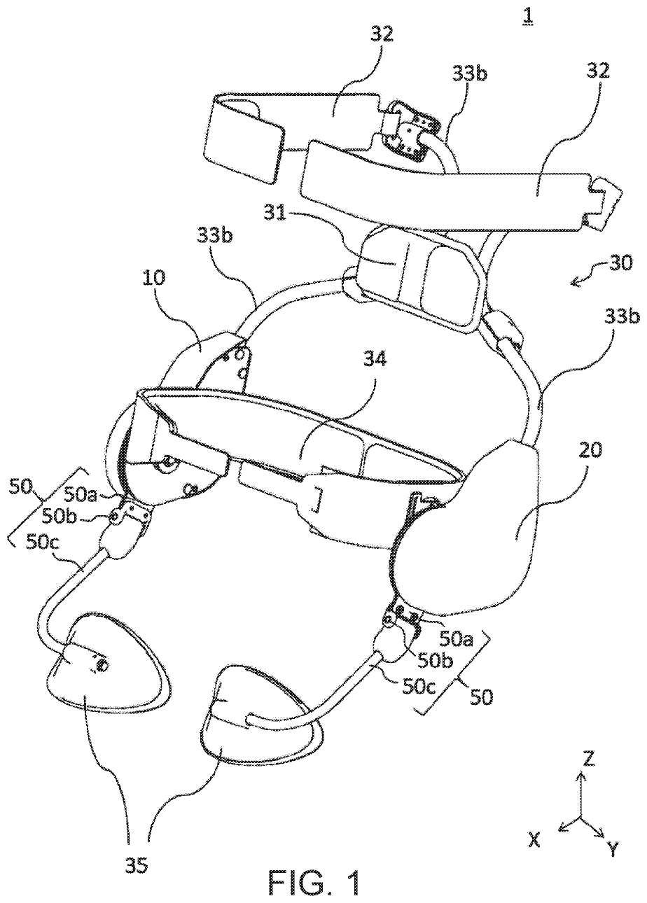

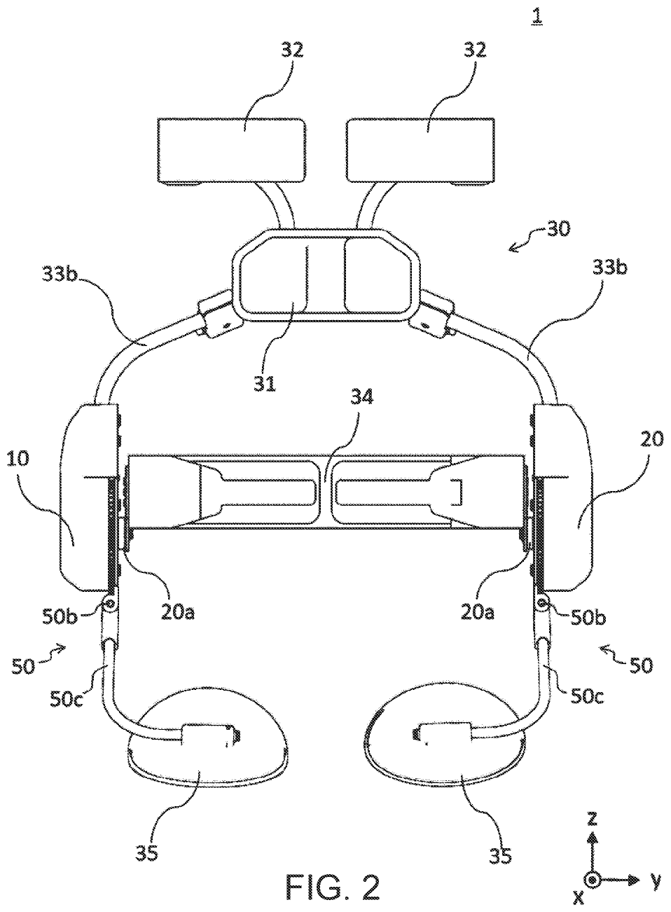

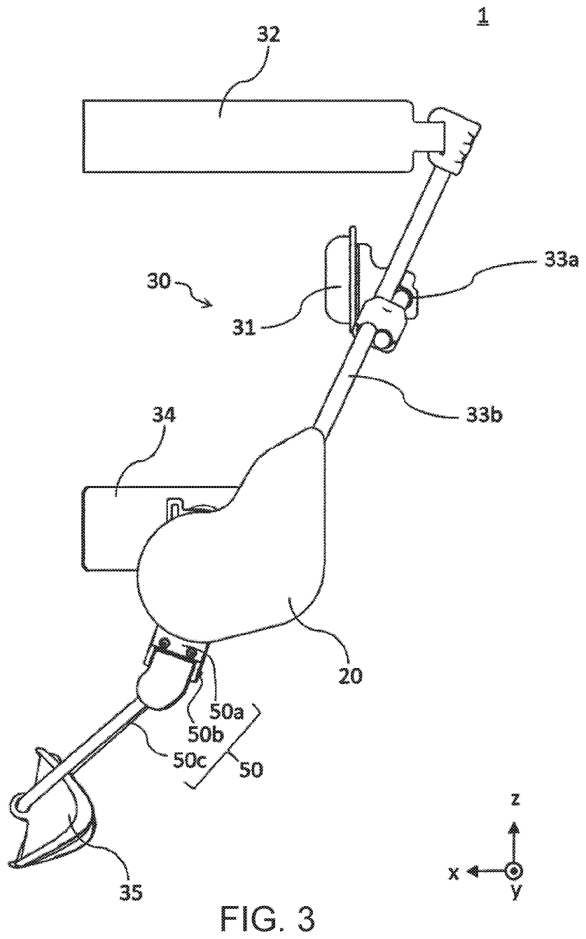

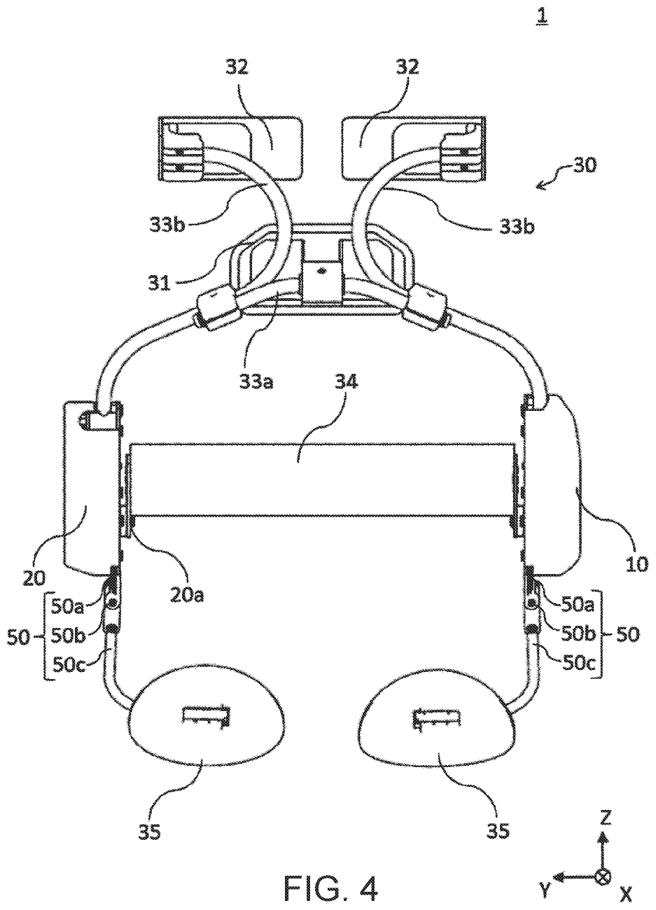

FIG. 1 is a perspective view of the device 1. FIG. 2 is a front view thereof. FIG. 3 is the right side view. FIG. 4 is the back view. The device 1 is attached so as to cover the back of a user from both sides of the user. The front side of the user and the front surface of the device 1 are in the same direction. In the following, the direction from the back side to the front side of the user is defined as a +X direction, the direction from the right side to the left side of the user is defined as a +Y direction, and the direction from the lower side to the upper side of the user is defined as a +Z direction.

As illustrated in FIGS. 1 to 4, the device 1 mainly includes an assisting unit 10 attached to the right side of the user, an assisting unit 20 attached to the left side of the user, an attachment part 30 that is a member for attaching the assisting units 10, 20 to the user, and a lower limb rod 50.

The attachment part 30 mainly includes a back pad part 31, a chest pad part 32, connection parts 33a, 33b, a waist fixing member 34, and a pad 35.

The back pad part 31 is a flat plate to be in contact with the back of the user when attached. In the part 31, a surface to be in contact with the back of the user (that is, the front side surface of the device 1) is made of an elastic soft material. Therefore, the part 31 is comfortable without biting into the back when attached. The shape of the part 31 is not limited to the form illustrated in the drawings.

The chest pad part 32 (corresponding to a chest fixing member of the invention) is a member fixed to the chest of the user when attached. In the embodiment, the part 32 includes a pair of band-shaped members made of an elastic soft material so as to be deformable following the motion of the user. Connection parts (not illustrated) are provided at both ends on the front surface side of the part 32. The connection parts connect the pair of band-shaped members, thereby keeping the part 32 in a cylindrical shape. An adjustment mechanism such as a belt (not illustrated) allows adjustment of the inner peripheral length of the part 32 so that the part 32 comes into contact with the chest of the user.

The connection parts 33a, 33b are symmetrical members mutually connecting the assisting units 10, 20, the back pad part 31, and the chest pad part 32. The parts 33a, 33b are formed by curving a rod material such as a hollow round rod. The parts 33a, 33b are rigid bodies, and fix the relative position relation of the units 10, 20, the part 31, and the part 32. Particularly, because the part 31 and the parts 33a, 33b are rigid bodies, the unit 10 and the assisting unit 20 are connected and fixed by the part 31 and the parts 33a, 33b. Therefore, it is possible to prevent the torsion of the units 10, 20 and securely transmit the assisting force of the units 10, 20 to the user even if certain force generates for causing mutual torsion of the unit 10 and the unit 20.

The connection part 33a is a rod-like member provided on the back surface side of the back pad part 31, and fixes the part 31 and a pair of connection parts 33b. The part 33a is curved to be convexly upward. The connection parts 33b are members extending upward and downward from the part 31, and the upper ends of the assisting units 10, 20 are individually connected to the lower ends of the parts 33b.

The chest pad part 32 is connected to each of the pair of connection parts 33b. The part 32 is formed to be open to the right and left sides, viewed from the front surface of the device 1. The user carries the device 1 along the back in the state where the part 32 is open to the right and left sides, and then closes the part 32 in front of the body so that the inner side surface of the part 32 comes in contact with the body.

The waist fixing member 34 is a single band-shaped member, and is connected to the assisting units 10, 20. The member 34 keeps the right and left assisting units 10, 20 around the waist of the user. A hook and loop fastener, for example, is fixed on both ends on the front surface side of the waist member 34, and the connecting force of the hook and loop fastener keeps the member 34 in a cylindrical shape. The inner peripheral length of the member 34 is adjustable by changing a connection area of the hook and loop fastener, so that the member 34 comes into contact with the waist of the user. The member 34 is a soft member, and is deformable following the motion of the user. Thus, the user is able to move easily.

The assisting units 10, 20, the chest pad part 32, and the waist fixing member 34 are connected by the connection part 33, and the units 10, 20 are fixed to the user by the part 32 and the member 34. Therefore, even if the tensile force of the cord-like body generates force causing torsion of the units 10, 20, it is possible to prevent torsion thereof, and to smoothly rotate the lower limb rod 50 following the rotary motion of the femoral region.

The form of attaching the assisting units 10, 20 to the sides of the user is not limited to the above-described form. For example, the waist fixing member may be made of a hard material, and the units 10, 20 may be attached to the sides of the user using the nondeformable waist fixing member. However, the units 10, 20 are preferably attached to the sides of the user using the part 32 and the member 34 for preventing torsion of the units 10, 20 and securely transmitting assisting force to the user.

The form of the attachment part 30 is also not limited to the above-described form. For example, the height positions (in z direction) of the back pad part 31 and the chest pad part 32 may be substantially same. Moreover, the back pad part 31 may be omitted, and the chest pad part 32 may be a single band-shaped member.

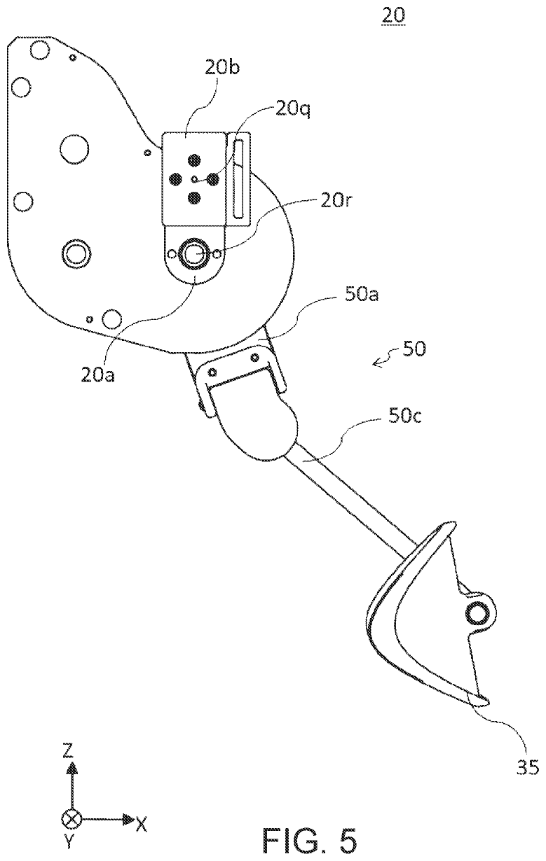

FIG. 5 is a left side view schematically illustrating the assisting unit 20. A connection part 20a is connected to the left side surface of the unit 20. The connection part 20a is a flat plate-shaped member. A rotation axis 20r extends through the unit 20 and the connection part 20a, and the unit 20 and the connection part 20a are rotatable with the rotation axis 20r as a center.

A flat plate-shaped connection part 20b is provided at the connection part 20a. The waist fixing member 34 is provided at the connection part 20b. A rotation axis 20q extends through the connection part 20a and the connection part 20b, and the connection part 20a and the connection part 20b are rotatable with the rotation axis 20q as a center. In other words, the member 34 is provided at the connection part 20a to be rotatable with the rotation axis 20q as a center.

With the connection part 20a interposed between the assisting unit 20 and the waist fixing member 34, it is possible to arrange the rotation axis 20r for the connection part 20a and the assisting unit 20 at a different position from the rotation axis 20q for the connection part 20a and the member 34. Here, the rotation axis 20q is offset to the +Z axis direction from the rotation axis 20r. In this manner, the rotary motion of one of the unit 20 and the member 34 is not transmitted directly to the other, thereby enabling the unit 20 and the member 34 to rotate independently. Moreover, the unit 20 is rotated in accordance with an angle of a femoral region, while the member 34 is rotated in accordance with an angle of a waist part. In a human body, the height in the Z-axis direction is different between the rotation axis of a femoral region and the rotation axis of a waist. Therefore, if the rotation axis of the unit 20 is arranged at the substantially same height position as the rotation axis of the member 34, the motion assisting device 1 cannot follow the forward leaning motion of a human body, thereby causing torsion of the member 34. On the other hand, the rotation axis 20r is arranged at a different position from the rotation axis 20q, so that the position of the 20r substantially matches the rotation position of the femoral region of the user, and so that the position of the 20q substantially matches the rotation position of the waist of the user. This allows the device 1 to follow the forward bending motion of the human body more easily.

The description will be returned to FIGS. 1 to 4. The pad 35 is a curved member covering the front side of the femoral region of the user. The pad 35 is provided at the lower limb rod 50. The user brings the pad 35 into contact with the front side of the femoral region, whereby the motion of the femoral region is transmitted to the assisting units 10, 20.

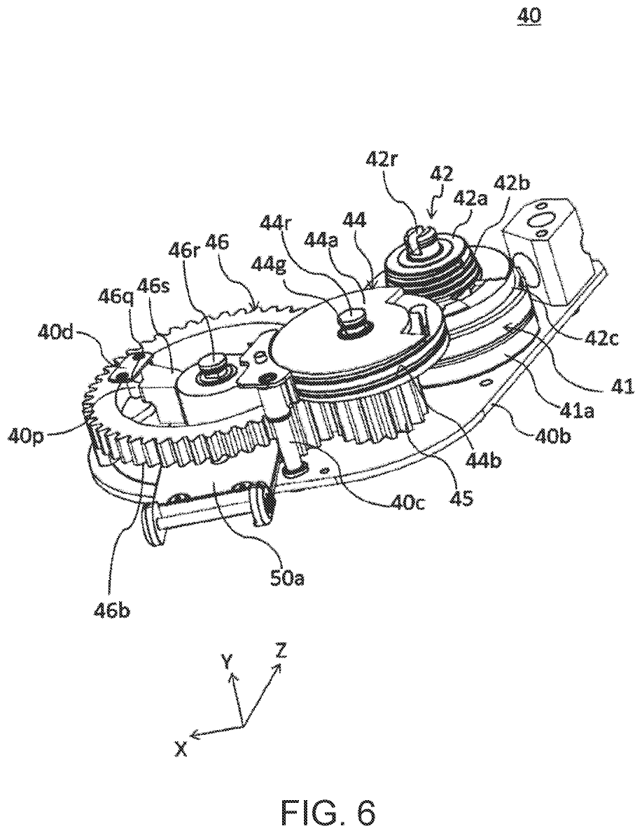

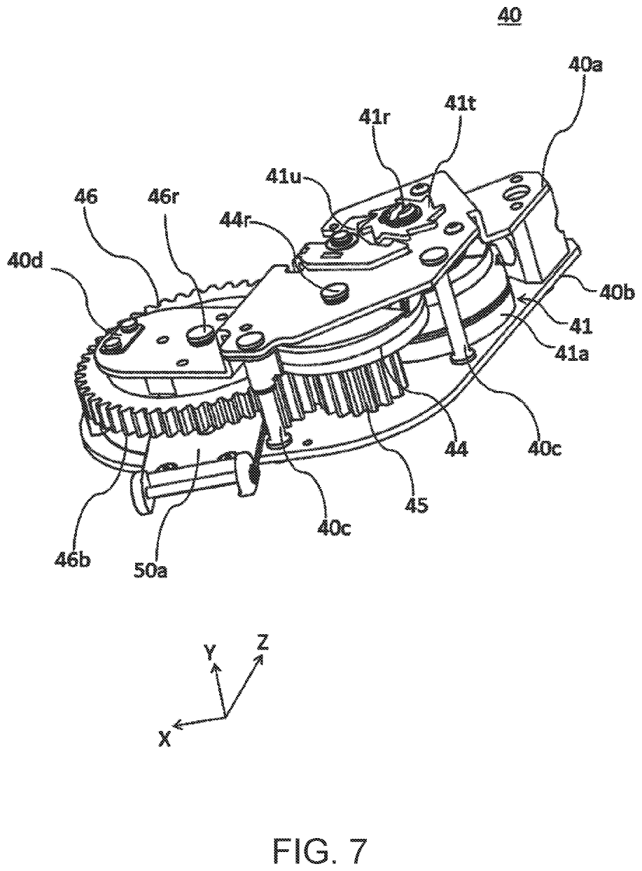

The lower limb rod 50 is provided substantially along the femoral region of the user, and can be attached to the femoral region of the user through the pad 35. The rod 50 is connected to a rotation axis 46r (see FIGS. 6, 7 and the like), and is rotatable with the axis 46r as a center.

The lower limb rod 50 includes a lower limb rod connection part 50a. One end of the lower limb rod connection part 50a is connected to the rotation axis 46r. A hinge 50b is provided at the other end of the part 50a. A pad support body 50c is connected to the part 50a through the hinge 50b. The hinge 50b has an axis in a direction substantially orthogonal to the longitudinal direction of the part 50a. Therefore, the movement in the +X axis direction of the pad 35 connected to the distal end of the body 50c is transmitted to the part 50a. Moreover, the movement in the +Y axis direction of the pad 35 is converted into rotation of the hinge 50b, and does not rotate the part 50a.

The following will describe the assisting units 10, 20. Because the units 10 unit 20 are mutually symmetrical, the following description will be given using the assisting unit 20.

FIGS. 6 and 7 are perspective views schematically illustrating the power unit 40 of the assisting unit 20. FIG. 6 illustrates the state when viewed from the −Z axis direction to the +Z axis direction. FIG. 6 omits a first cover 40a. FIG. 7 is a perspective view when viewed from the same direction as in FIG. 6, and illustrates the state where the cover 40a is attached. FIGS. 6 and 7 illustrate a part of the lower limb rod 50.

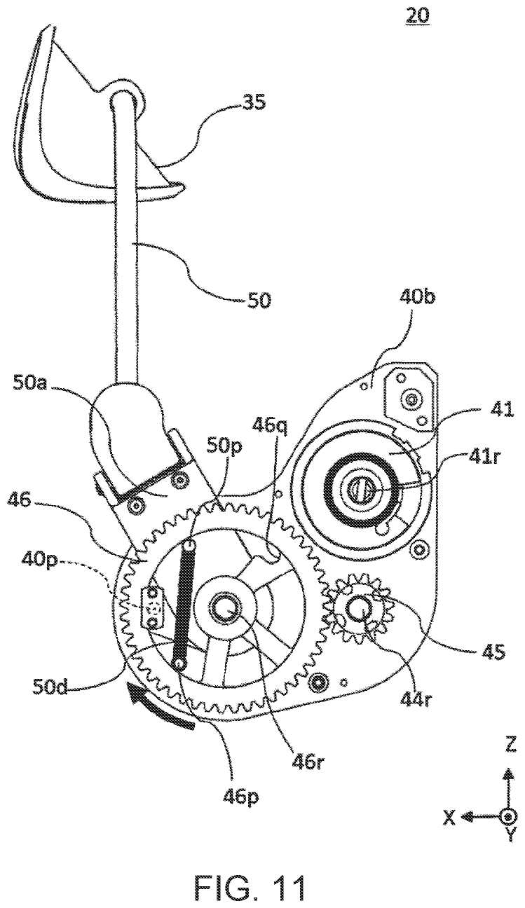

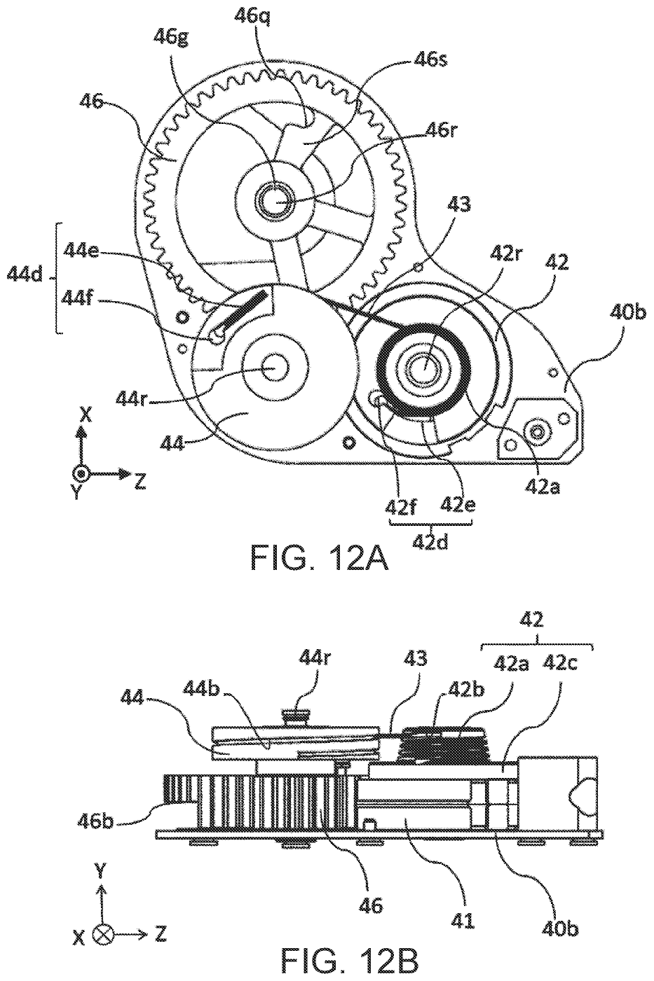

The power unit 40 mainly includes the first cover 40a, a second cover 40b, an elastic body unit 41, the first pulley 42, the cord-like body 43 (see FIGS. 11, 12, and the like), the second pulley 44, a small gear 45, and the large gear 46.

The first cover 40a and the second cover 40b are substantially flat plates forming the outer edges on the +Y side and −Y side, respectively, of the power unit 40. The covers 40a and 40b face to each other with space therebetween. Both ends of a plurality of cylindrical columnar parts 40c are connected individually to the covers 40a and 40b, and the parts 40c keep the space between the covers 40a and 40b. The elastic body unit 41, the first pulley 42, the cord-like body 43, the second pulley 44, the small gear 45, the large gear 46, and a part of the lower limb rod connection part 50a are provided between the cover 40a and 40b.

The elastic body unit 41 includes a case 41a, and an elastic body (not illustrated) held in the case 41a. The elastic body is a spiral spring formed by winding a plate-shaped member in the surface direction into a spiral shape. Although the spiral spring is used as an elastic body in the embodiment, the form of the elastic body is not limited thereto.

The first pulley 42 mainly includes a substantially truncated cone-shaped pulley main body 42a and a rotation axis 42r. The pulley 42 is rotatable with the axis 42r as a center together therewith. The outer periphery of the body 42a has a shape gradually tapered toward the distal end. The outer periphery of the body 42a has a helical groove 42b corresponding to the diameter of the cord-like body 43.

The first pulley 42 is provided on the case 41a of the elastic body unit 41 through a base 42c. The rotation axis 42r projects into the case 41a, although this is not illustrated. One end of the elastic body is provided at the axis 42r, and the other end thereof is provided at the case 41a. Therefore, when the pulley 42 is rotated, the elastic body energizes force in accordance with a rotation angle of the pulley 42 thereto.

As illustrated in FIG. 7, the rotation axis 41r of the first pulley 42 is provided with a ratchet gear 41t on the outer side (+Y side) of the first cover 40a. Moreover, a ratchet receiving part 41u engaged with teeth of the ratchet gear 41t is provided on the first cover 40a. The part 41u restricts rotation of the gear 41t and the axis 41r in the counterclockwise direction. With the gear 41t and the axis 41r, the elastic body is wound and tightened by an amount corresponding to a certain number of rotation even in the initial state (state where a load is not applied on the large gear 46 by the lower limb rod 50), and the wound and tightened state is kept. This allows adjustment of the strength of energizing force of the elastic body.

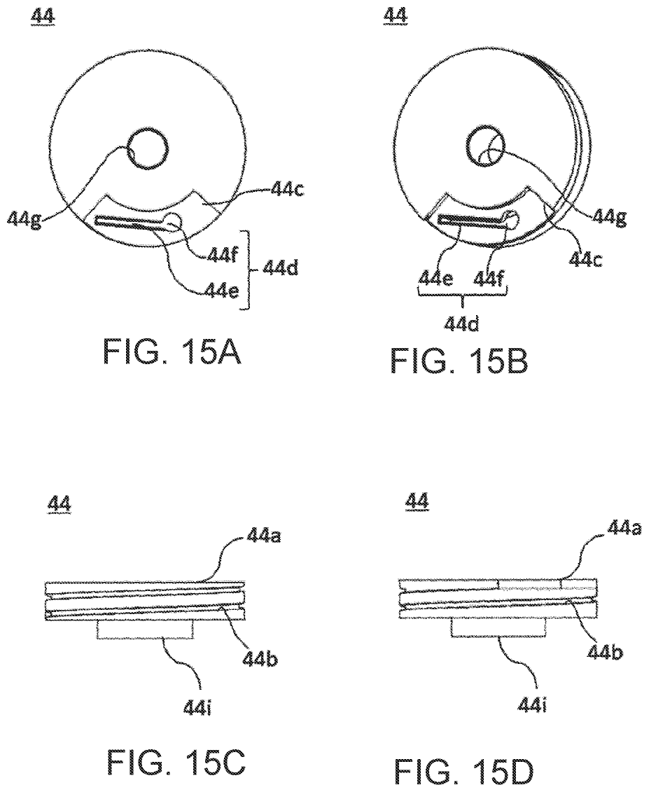

The second pulley 44 mainly includes a substantially disk-shaped pulley part 44a whose outer periphery is wound by the cord-like body 43, and a thin columnar projection part 44i (see FIGS. 15C-15D and the like) provided on a part 44a on the surface facing the −Y side of the part 44a. The outer periphery of the part 44a has a helical groove 44b corresponding to the diameter of the body 43. A through hole 44g into which an axis 44r extends is provided in substantially the center of the part 44a and the projection part 44i. The pulley 44 is rotatable with the axis 44r as a center.

The cord-like body 43 is housed in the groove 42b and the groove 44b so that the first end of the body 43 is provided at the first pulley 42 and the second end thereof is provided at the second pulley 44. The arrangement and the like of the body 43 will be specifically described later.

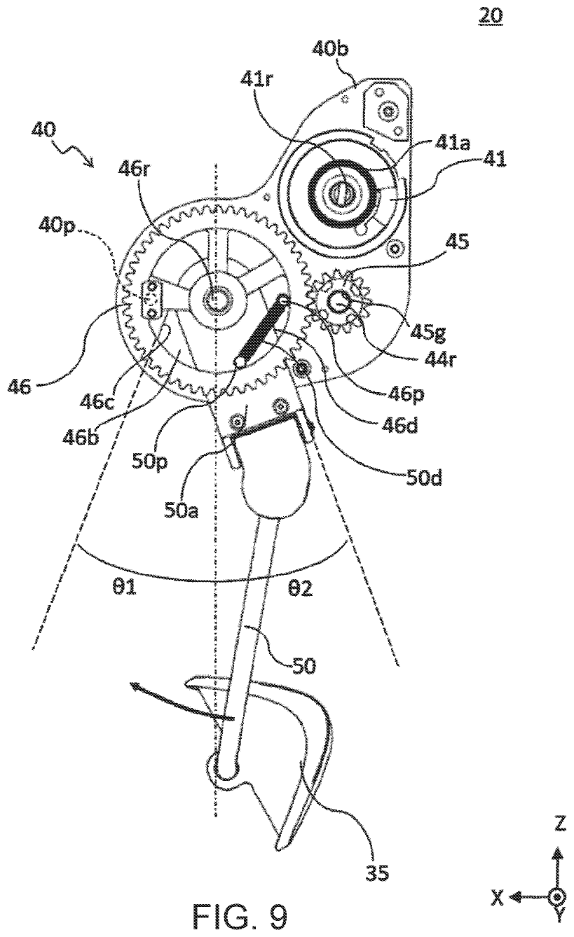

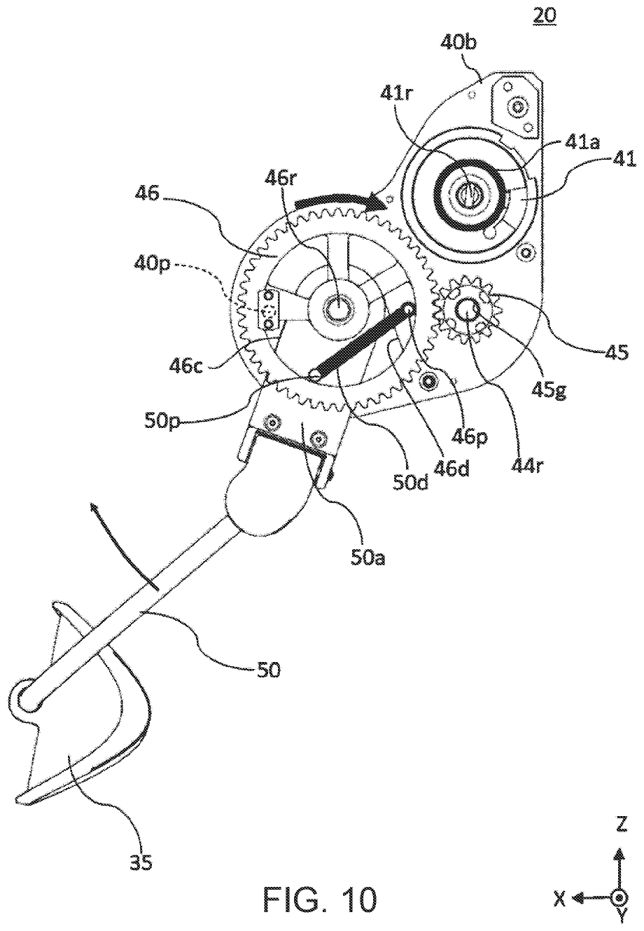

The small gear 45 is provided at the second pulley 44, and includes, in substantially the center thereof, a through hole 45g (see FIGS. 9, 10, and the like) into which the rotation axis 44r extends. The gear 45 is rotated together with the pulley 44 with the axis 44r as a center.

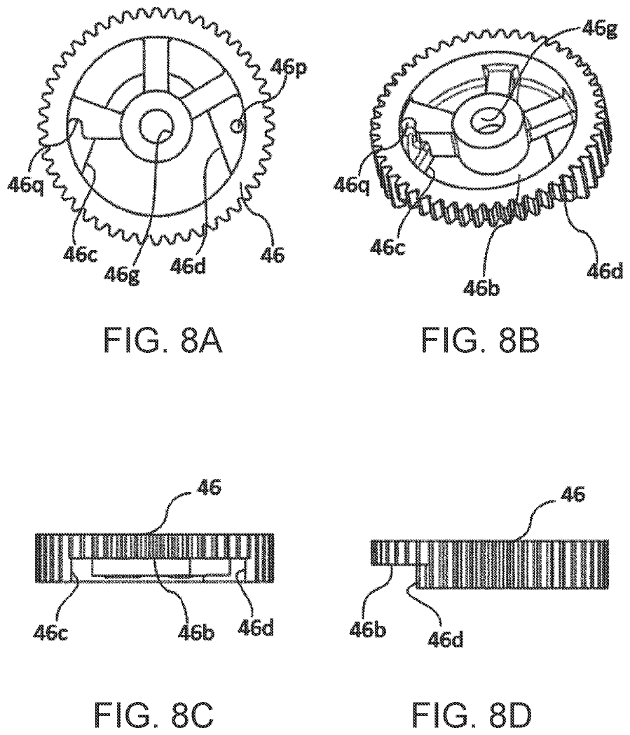

The large gear 46 is a gear having a larger number of teeth than the small gear 45. The gear 46 includes, in substantially the center thereof, a through hole 46g (see FIGS. 8A-8B) into which the rotation axis 46r extends, and is rotatable with the axis 46r as a center. The gear 46 can be fitted to the gear 45, and rotates along with the rotation of the gear 45.

A stationary plate 40d is screwed to the first cover 40a. A large gear fixing pin 40p is provided on the plate 40d. As illustrated in FIG. 6, the pin 40p projects to a direction toward the cover 40b (to the −y side) through a hole (not illustrated) formed on the cover 40a.

A pin receiving part 46q is provided at a spoke 46s of the large gear 46. The part 46q is a substantially columnar-shaped recessed portion, and extends along the Y direction. The large gear fixing pin 40p can be fitted to the part 46q. In this manner, it is possible to restrict the rotation of the gear 46 in the counterclockwise direction, viewed from the +Y direction.

FIGS. 8A-8D are diagrams schematically illustrating the large gear 46, in which FIG. 8A is a plan view viewed from the +Y direction, FIG. 8B is a perspective view, and FIGS. 8C-8D are side views. A recess 46b having a substantially fan shape with the rotation axis 46r as a center (the center of the arc portion of the recess 46b) is provided on the −Y side surface of the gear 46 (the surface to which the lower limb rod 50 is connected). The recess 46b includes side walls 46c, 46d.

The description will be returned to FIGS. 6 and 7. The lower limb rod connection part 50a is an elongate flat plate-shaped member thinner than the depth of the recess 46b, and is rotatable in the recess 46b along the flat surface of the bottom part of the recess 46b. When the part 50a comes into contact with the side wall 46c, rotation of the rod 50 can be transmitted to the gear 46, and the rotation of the gear 46 can be transmitted to the rod 50.

FIG. 9 is a diagram illustrating a state of the motion assisting device 1 (assisting unit 20) when a leg of the user is positioned rearward during walking. FIG. 10 is a diagram illustrating a state of the device 1 (unit 20) when a waist of the user is slightly bent. FIG. 11 is a diagram illustrating a state of the device 1 (unit 20) when the waist of the user is further bent. Note that FIG. 9 to FIG. 11 omit the cord-like body 43 and the second pulley 44.

The recess 46b is formed to be larger than the lower limb rod connection part 50a so that the part 50a is rotatable in the recess 46b. In the state illustrated in FIG. 9, the part 50a is positioned on the most −X side so as to be contact with the side wall 46d.

A pin 46p projecting to the +Y direction is provided at the large gear 46. Moreover, a pin 50p projecting to the +Y direction is provided at the lower limb rod connection part 50a. A lower limb rod energizing member 50d (a tension coil spring, for example) is provided between the pin 50p and the pin 46p, and energizes the lower limb rod connection part 50a in a direction where the part 50a is brought into contact with the side wall 46d (the direction where the pad 35 is pressed against a leg of the user). In the state illustrated in FIG. 9, the large gear fixing pin 40p is fitted to the pin receiving part 46q (not illustrated in FIGS. 9 and 10), and thus the large gear 46 does not rotate counterclockwise, when viewed from the +Y direction.

Between the side walls 46c, 46d, the lower limb rod connection part 50a is idly (freely, without resistance) rotatable in the recess 46b. When the user starts to bend the waist from the state illustrated in FIG. 9, the part 50a idly rotates clockwise in the recess 46b (see the arrow of FIG. 9). When the part 50a rotates by a given angle or larger, the part 50a comes into contact with the side wall 46c, as illustrated in FIG. 10.

When the user further bents the waist from the state illustrated in FIG. 10, and the lower limb rod connection part 50a rotates further forward (clockwise in FIG. 10, see the arrow thereof), the part 50a presses against the side wall 46c, and applies force in the rotation direction onto the large gear 46 to rotate the gear 46. As a result, the gear 46 rotates clockwise (see the bold arrow of FIG. 10), and the large gear fixing pin 40p is released from the pin receiving part 46q, resulting in the state illustrated in FIG. 11. That is, the transmission mechanism formed by the recess 46b, the side wall 46c, and the part 50a enables switching between the first form in which the part 50a does not rotate the gear 46 and the second form in which the part 50a applies force in the rotation direction to the gear 46 and rotates the gear 46.

The angle for switching between the first form and the second form and the idling angle of the lower limb rod 50 are determined by the side walls 46c and 46d. As illustrated in FIG. 9, the side wall 46c determines the angle for switching between the first form and the second form on the front side of the user. Moreover, the side walls 46c, 46d define the idling angle of the lower limb rod 50 on the front side and the back side of the user. In the embodiment, an angle θ1 formed by a center line and the side wall 46c and an angle θ2 formed by the center line and the side wall 46d are substantially 20°. However, the angles θ1, θ2 are not limited to the illustrated forms as long as the angle θ1 is larger than the angle θ2. When the angle θ1 is larger than the angle θ2, the angle for switching between the first form and the second form can be an angle following the walking motion.

The description will be returned to FIG. 11. Along with the rotation of the large gear 46, the small gear 45 fitted to the large gear 46 rotates. Then, the second pulley 44 rotates together with the gear 45. Along with the rotation of the pulley 44, the cord-like body 43 is drawn out from the first pulley 42, thereby rotating the pulley 42.

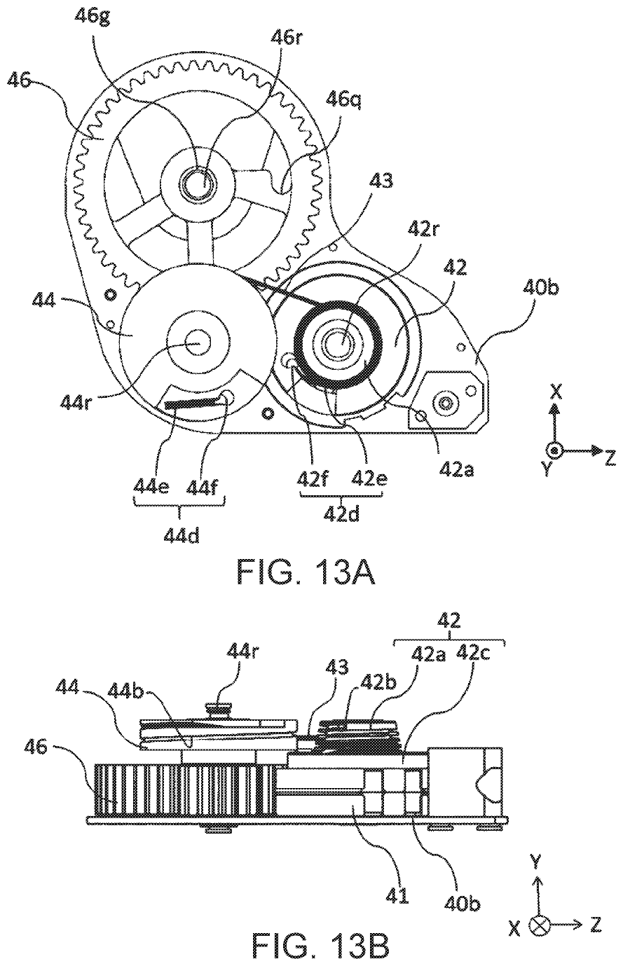

Here, there will be described a configuration of rotating the first pulley 42 along with the rotation of the second pulley 44. FIGS. 12A-13B are diagrams illustrating the states of the pulley 42 and 44, and the cord-like body 43. FIGS. 12A-12B illustrate the state where the body 43 is mainly wound around the pulley 42. FIGS. 13A-13B illustrate the state where the body 43 is wound around the pulley 42 and 44.

The cord-like body 43 is a wire rope, for example, and the first end of the body 43 is connected to the first pulley 42, and the second end thereof is connected to the second pulley 44. Although a wire rope is used as the body 43 in the embodiment, a string, a steel wire, or the like may be used as the body 43. A locking member (not illustrated) having a larger diameter than a halfway part of the body 43 is connected to both ends of the body 43.

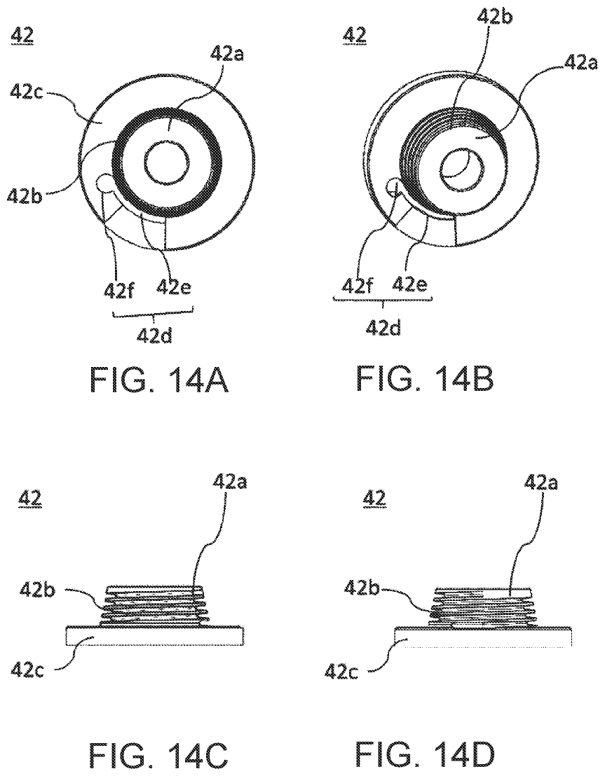

FIGS. 14A-14D are diagrams schematically illustrating the first pulley 42, in which FIG. 14A is a plan view viewed from the +Y direction, FIG. 14B is a perspective view, and FIGS. 14C-14D are side views. The pulley 42 is provided with a cord-like body attachment part 42d. The part 42d mainly includes a long hole 42e and a circular hole 42f. The locking member is fitted into the circular hole 42f and fixed by caulking or the like, whereby one end of the body 43 is locked by the pulley 42. The body 43 has its end fixed to the part 42d, and is stored as it is in the groove 42b and wound helically around the outer periphery of the pulley main body 42a.

FIGS. 15A-15D are diagrams schematically illustrating the second pulley 44, in which FIG. 15A is a plan view viewed from the +Y direction, FIG. 15B is a perspective view, and FIGS. 15C-15D are side views. The pulley 44 includes a fan-shaped recess 44c, and a cord-like body attachment part 44d is provided at the recess 44c. The part 44d mainly includes a large hole 44e and a circular hole 44f. The locking member is fitted into the hole 44f and fixed by caulking or the like, whereby one end of the cord-like body 43 is locked by the pulley 44. The body 43 has its end fixed to the part 44d, and is stored as it is in the groove 44b and wound helically around the outer periphery of the part 44a.

The description will be returned to FIGS. 12A-13B. In FIGS. 12A-12B, the second pulley 44 is not rotated. Thus, the cord-like body 43 is wound around the substantially entire first pulley 42, and is hardly wound around the second pulley 44 (the winding of the body 43 around the pulley 44 is about substantially ¼ of the periphery). Then, when the pulley 44 is rotated, the body 43 is unwound from the pulley 42 and then wound around the pulley 44, as illustrated in FIGS. 13A-13B.

Each of the grooves 42b, 44b is helical, and the cord-like body 43 is arranged to be orthogonal to the rotation axis 42r and the rotation axis 44r when the first pulley 42 and the second pulley 44 are viewed from the side surface. Therefore, it is possible to prevent the body 43 from being drawn out diagonally between the pulley 42 and the pulley 44, smoothen sliding of the body 43, and thus suppress a loss in transmission of force.

When the second pulley 44 is rotated, the cord-like body 43 rotates the first pulley 42. Because the elastic body is provided in the pulley 42, the pulleys 44 and 42 are rotated against the energizing force of the elastic body. As the rotation amount of the pulley 42 is increased, the elastic force of the elastic body is increased. Along with this, the outer diameter of the pulley main body 42a of the pulley 42 around which the body 43 is wound becomes larger. Therefore, it is possible to keep force (an apparent spring constant) required to unwind the body 43 from the pulley 42 (draw out the body 43 from the pulley 42) substantially constant.

The outer diameter of the second pulley 44 is larger than the outer diameter of the first pulley 42. Thus, when the energizing force of the elastic body is transmitted from the pulley 42 to the pulley 44, a torque is amplified between the pulley 42 and the pulley 44. Moreover, the outer diameter of the small gear 45 is smaller than the outer diameter of the pulley 44, and the gear 45 is engaged with the large gear 46 having a larger number of teeth. Thus, a torque is amplified between the pulley 44 and the gear 45, and the gear 46. Therefore, it is possible to apply assisting force larger than the energizing force of the elastic body to the lower limb rod 50.

The description will be returned to FIGS. 9 to 11. When the user moves in a direction of bending the waist, the lower limb rod 50 and the large gear 46 rotate clockwise (see the arrows of FIG. 9 to FIG. 11) against the energizing force of the elastic body. Along with the rotation of the gear 46, the small gear 45 and the second pulley 44 rotate against the energizing force of the elastic body. Moreover, along with the rotation of the pulley 44, the cord-like body 43 is unwound from the first pulley 42 and then wound around the pulley 44, whereby the pulley 42 rotates against the energizing force of the elastic body.

When the user moves in a direction of stretching the waist, the elastic body is subjected to restoring force of drawing the drawn-out cord-like body 43 into the original position. That is, along with the rotation of the first pulley 42 by energizing force of the elastic body, the body 43 is wound around the pulley main body 42a and unwound from the second pulley 44, whereby the body 43 rotates the pulley 44. Along with the rotation of the pulley 44, each of the small gear 45 and the large gear 46 is rotated, whereby the lower limb rod 50 is restored to the original position.

In the embodiment, it is possible to assist a motion of stretching the waist at the time of lifting the body from the forward bending posture with a simple configuration without using external power such as electricity. Moreover, the energizing force of the elastic body can be amplified in two stages, thereby allowing energization of the lower limb rod 50 with larger force than the energizing force of the elastic body. Therefore, it is possible to generate a larger assisting force than the energizing force of the elastic body, and efficiently assist a motion of restoring the waist of the user to the upright state.

In the embodiment, the lower limb rod 50 rotates idly while the rod 50 moves within a given angle, such as while the user is walking, and thus no assisting force is generated. Therefore, the motion of the user is not impeded while walking. Moreover, the presence and absence of assistance is switched in accordance with a relative angle between the rod 50 and the large gear 46. Thus, it is not necessary to perform a switching operation of whether the assisting force is to be generated, which improves usability.

### Second Embodiment

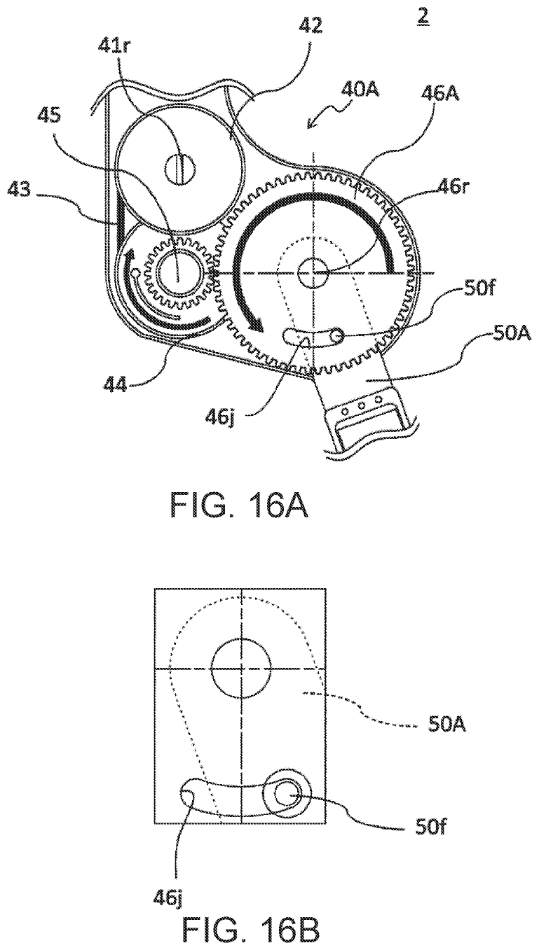

Regarding the second of the disclosed embodiments, the following will mainly describe a part different from the first embodiment. The same configurations as the first embodiment are represented with the same reference signs in the following description. FIGS. 16A-16B are longitudinal sectional views schematically illustrating the power unit 40A of the motion assisting device 2 according to the second embodiment, in which FIG. 16A is an entire view of the power unit 40A, and FIG. 16B is a partially enlarged view thereof.

A large gear 46A includes a long hole 46j curved in an arc shape with the rotation axis 46r as a center (the center of the circle about which the arc is formed). Moreover, a lower limb rod 50A includes a pin 50f fitted to the long hole 46j. When the lower limb rod 50A rotates, the pin 50f moves in the long hole 46j. While the pin 50f moves in the hole 46j, the gear 46A does not rotate even when the rod 50A rotates (first form), thereby generating no force of assisting the motion.

When a rotation angle of the lower limb rod 50A becomes a given angle or larger, the pin 50f comes into contact with an end of the long hole 46j. As a result, the pin 50f presses against the end of the hole 46j so that the rod 50A rotates, whereby the large gear 46A also rotates (second form). Moreover, the end of the hole 46j presses against the pin 50f so that the large gear 46A rotates, whereby the rod 50A also rotates (second form). An angle for switching between the first form and the second form and an idling angle of the rod 50 are defined by the length of the hole 46j. In the embodiment, the transmission mechanism is achieved by the pin 50f and the hole 46j.

### Third Embodiment

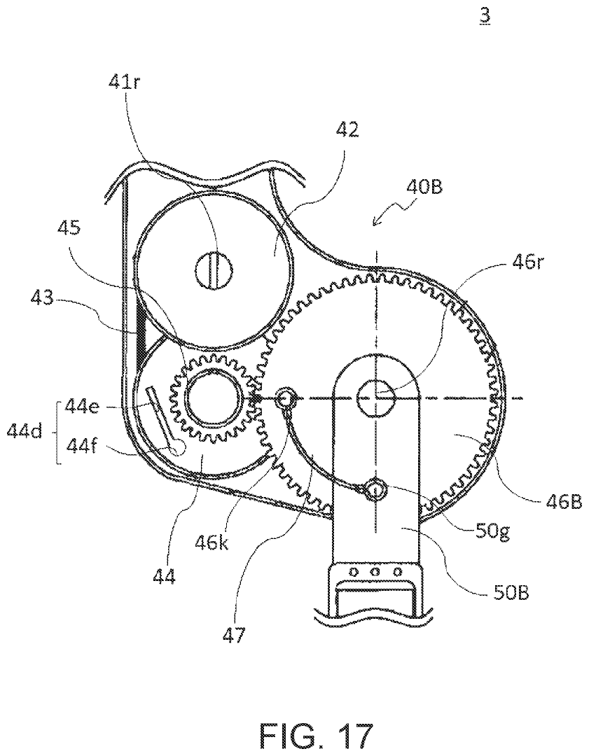

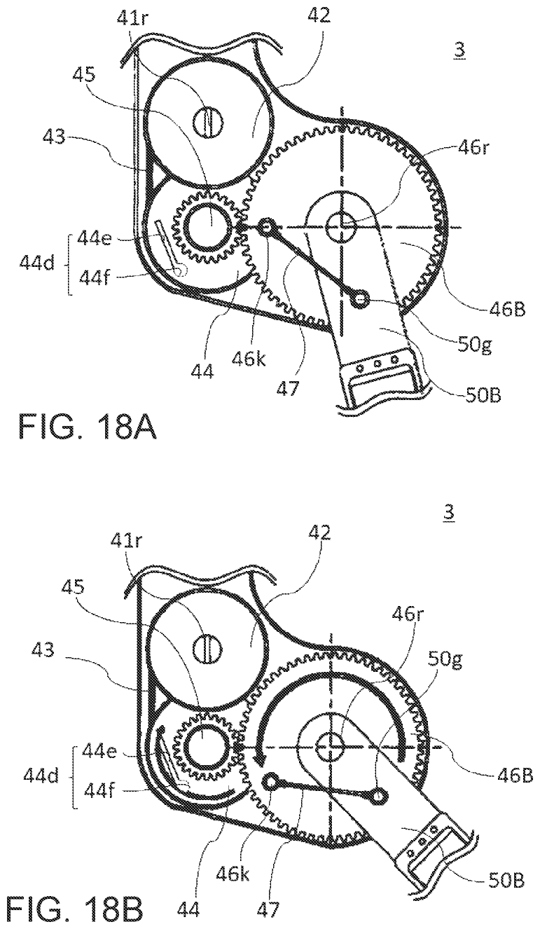

Regarding the third of the disclosed embodiments, the following will mainly describe a part different from the first and second embodiments. FIGS. 17-18B are longitudinal sectional views schematically illustrating the power unit 40B of the motion assisting device 3 according to the third embodiment. FIG. 17 illustrates a state where the user stands upright. FIG. 18A illustrates a state where the user is walking, and FIG. 18B illustrates a state where the user bends the waist.

The motion assisting device 3 includes a second cord-like body 47 connecting a lower limb rod 50B and a large gear 46B. The rod 50B rotates idly within a range where the body 47 is not stretched. Therefore, when the body 47 is not stretched, the gear 46B does not rotate even when the rod 50B rotates (first form), thereby generating no force of assisting the motion.

When the lower limb rod 50B moves forward (right direction of FIGS. 17-18B), and a fixing portion 46k between the second cord-like body 47 and the large gear 46B is separated from a fixing portion 50g between the body 47 and the rod 50B by a given distance or larger, the rod 50B draws the body 47 so that the gear 46B rotates, and the gear 46B draws the body 47 so that the rod 50B rotates (second form). The angle for switching between the first form and the second form and the idling angle of the rod 50 are defined by a length of the body 47 and a distance between the portions 46k, 50g at both ends of the body 47. In the embodiment, the transmission mechanism is achieved by the body 47.

Embodiments of the invention have been specifically described above with reference to the drawings. However, the concrete configurations are not limited to the disclosed embodiments, and include design changes and the like within a range not departing from the scope of the invention. The technical ideas of the disclosed embodiments are also applicable to any other than a motion assisting device assisting a motion of a human body.

Moreover, in the invention, “substantially” means a concept including not only a strictly identical case but also a case with an error or deformation within a range not losing the functionality achieved in the strictly identical case. For example, substantially parallel and substantially orthogonal are not limited to the cases of strictly parallel and strictly orthogonal, respectively. Moreover, even the simple expression of parallel, orthogonal, or the like, includes not only the case of strictly parallel, strictly orthogonal, or the like, but also the case of substantially parallel, substantially orthogonal, or the like. Furthermore, in the invention, “vicinity” is a concept indicating, when used in “the vicinity of A”, for example, closeness to A including or excluding A.

### REFERENCE SIGNS LIST

1, 2, 3: motion assisting device
10, 20: assisting unit
20a, 20b: connection part
20q, 20r: rotation axis
30: attachment part
31: back pad part
32: chest pad part
33: connection part
33a, 33b: connection part
34: waist fixing member
35: pad
40, 40A, 40B: power unit
40a: first cover
40b: second cover
40c: columnar part
40d: stationary plate
40p: large gear fixing pin
41: elastic body unit
41a: case
41r: rotation axis
41t: ratchet gear
41u: ratchet receiving part
42: first pulley
42a: pulley main body
42b: groove
42c: base
42d: cord-like body attachment part
42e: long hole
42f: circular hole
42r: rotation axis
43: cord-like body
44: second pulley
44a: pulley part
44b: groove
44c: recess
44d: cord-like body attachment part
44e: long hole
44f: circular hole
44g: through-hole
44i: projection part
44r: rotation axis
45: small gear
46, 46A, 46B: large gear
46b: recess
46c, 46d: side wall
46j: long hole
46k: fixing portion
46p: pin
46q: pin receiving part
46r: rotation axis
46s: spoke
47: second cord-like body
50, 50A, 50B: lower limb rod
50a: lower limb rod connection part
50b: hinge
50c: pad support body
50d: lower limb rod energizing member
50f: pin
50g: fixing portion
50p: pin

1. A motion assisting device configured to assist a motion of lifting an upper body of a user, the device comprising:
an assisting unit configured to be mounted to a lateral side of the user, the assisting unit including:
a first pulley having a first rotation axis and being rotatable with the first rotation axis as a center;
an elastic body provided at the first rotation axis and configured to transfer an energizing force to the first pulley in accordance with a rotation angle of the first pulley;
a cord-like body including a first end connected to the first pulley;
a second pulley to which a second end of the cord-like body is connected and that is rotatable with a second rotation axis as a center, the second pulley having an outer diameter larger than an outer diameter of the first pulley;
a small gear provided at the second pulley and rotating together with the second pulley with the second rotation axis as a center; and
a large gear having a larger number of teeth than the small gear, being fittable to the small gear, and being rotatable with a third rotation axis as a center,
a lower limb rod that is connected to the assisting unit at the third rotation axis and is attachable to a femoral region of the user; and
a transmission mechanism between the assisting unit and the lower limb rod and that transmits rotation of the large gear to the lower limb rod or transmits rotation of the lower limb rod to the large gear,
wherein:
the cord-like body is wound around an outer peripheral surface of the first pulley and an outer peripheral surface of the second pulley; and
when the energizing force of the elastic body is transmitted to the first pulley, the first pulley rotates so that the cord-like body is wound around the first pulley and the cord-like body is unwound from the second pulley, whereby the second pulley is rotated, the rotation of the second pulley rotates the small gear and the large gear, and the rotation of the large gear rotates the lower limb rod through the transmission mechanism.

2. The device according to claim 1, wherein:
the transmission mechanism is configured to perform switching between a first form and a second form in accordance with a relative angle of the lower limb rod and the large gear; and
when the lower limb rod is rotated:
in the first form, the lower limb rod does not apply force in a rotation direction to rotate the large gear so as to rotate the large gear, and
in the second form, the lower limb rod applies force in a rotation direction to the large gear so as to rotate the large gear.

3. The device according to claim 2, wherein an angle for switching between the first form and the second form on a front side of the user is equal to or larger than an angle for switching between the first form and the second form on a back side of the user.

4. The device according to claim 1, wherein:
the large gear includes a fan-shaped recess with the third rotation axis as a center on a surface to which the lower limb rod is connected;
the recess is larger than a connection part with the large gear of the lower limb rod in a rotation direction of the lower limb rod;
the lower limb rod is rotatable in the recess along a plane surface of a bottom part of the recess; and
rotation of the large gear is transmittable to the lower limb rod, and rotation of the lower limb rod is transmittable to the large gear, when the lower limb rod comes into contact with a side wall of the recess.

5. The device according to claim 1, wherein:
the lower limb rod includes a pin on a surface facing the large gear;
the large gear includes a long hole into which the pin is inserted;
the pin is movable in the long hole in accordance with rotation of the lower limb rod; and
rotation of the large gear is transmittable to the lower limb rod and rotation of the lower limb rod is transmittable to the large gear, when the pin comes into contact with an end of the long hole.

6. The device according to claim 1, further comprising:
a second cord-like body connecting the lower limb rod and the large gear,
wherein rotation of the large gear is transmittable to the lower limb rod and rotation of the lower limb rod is transmittable to the large gear, when the lower limb rod rotates in a direction separating a fixing portion between the second cord-like body and the large gear from a fixing portion between the second cord-like body and the lower limb rod by a given distance or larger.

7. The device according to claim 1, wherein:
the assisting unit includes:
a first assisting unit that is configured to be attached to a left side of the user; and
a second assisting unit that is configured to be attached to a right side of the user, and
the motion assisting device further comprises:
a chest pad that is configured to be fixed to a chest of the user;
a waist band that is configured to be fixed to a waist of the user; and
a connection part that connects the first assisting unit, the second assisting unit, the chest pad, and the waist band.

8. The device according to claim 7, further comprising:
an attaching member that is connected to the assisting unit and the waist band, wherein:
the attaching member is rotatable with respect to the assisting unit with a fourth rotation axis as a center, and is rotatable with respect to the waist band with a fifth rotation axis as a center; and
the fourth rotation axis and the fifth rotation axis are provided at mutually different positions.

9. The device according to claim 1, wherein:
the elastic body is a spiral spring in which a plate-shaped member is wound in a surface direction into a spiral shape;
the elastic body is provided at the first rotation axis;
an outer periphery of the first pulley is gradually tapered toward a distal end of the first pulley; and
the cord-like body is wound around the outer peripheral surface of the first pulley from a base of the first pulley toward a distal end of the first pulley.

10. The device according to claim 1, wherein:
a helical first groove is provided on the outer peripheral surface of the first pulley;
a helical second groove is provided on the outer peripheral surface of the second pulley;
the cord-like body is stored in the first groove and the second groove; and
the cord-like body is substantially orthogonal to the first rotation axis and the second rotation axis when the first pulley and the second pulley are viewed from a side surface.
